(19) United States
(12) Patent Application Publication
(10) Pub. No.: US 2013/0189346 A1
(43) Pub. Date: Jul. 25, 2013

(54) WOUND DRESSING

(71) Applicants: CHI SHENG CHU, NEW TAIPEI CITY (TW); CHIN-CHIEN HSIEH, NEW TAIPEI CITY (TW); HUNG-RU LIN, TAIPEI CITY (TW)

(72) Inventors: CHI SHENG CHU, NEW TAIPEI CITY (TW); CHIN-CHIEN HSIEH, NEW TAIPEI CITY (TW); HUNG-RU LIN, TAIPEI CITY (TW)

(73) Assignee: SILITECH TECHNOLOGY CORPORATION, NEW TAIPEI CITY (TW)

(21) Appl. No.: 13/354,648

(22) Filed: Jan. 20, 2012

(51) Int. Cl. A61K 9/70; A61P 31/00; A61K 38/27; A61P 17/02; A61P 29/00

(52) U.S. Cl. 424/445; 514/11.3

(57) ABSTRACT

The instant disclosure relates to a wound dressing, which includes a plurality of hydrogel particles and a dressing. The hydrogel particles are granular colloids capable of absorbing and releasing the fluid, and the hydrogel particles are disposed to fill up the wound entirely. The dressing is covered on the wound to seal the hydrogel particles inside the wound. Thus, the wound dressing can maintain the appropriate balance of moisture in the wound environment to promote healing.

### BACKGROUND OF THE INVENTION

1. Field of the Invention

The instant disclosure relates to a dressing; in particular, to a wound dressing which can be easily removed and capable of managing fluid in the wound to maintain an optimal healing environment.

2. Description of Related Art

A numbered of people are suffering from diabetes mellitus, often simply referred to as diabetes, in Taiwan. People with diabetes, other than the classical symptoms of polyuria (frequent urination), polydipsia (increased thirst) and polyphagia (increased hunger), the insufficient of collagens within the bodies will also impair the healing of the wound. What's worse, diabetic neuropathies is also the leading cause of amputation of feet, toes, legs, hands and arms among diabetes sufferers. People with diabetes are malfunctioned in their healing process, such as the inflammatory phase, proliferative phase, remolding phase, and epithelialization, and also a poor metabolism. Thus, often a small accidental cut may threaten the patients to amputation as the wounds can easily deteriorate into cavity wounds. In other words, a deep wound or a defect may have already occurred within the body, though the wound may seem just a small cut from the appearance. Therefore, if not properly taken care, or lack of necessary attention, the outcome of amputation may severely occur.

To promote healing, different types of dressings are researched. The major categories of moist or synthetic wound dressings currently in use are powder, foam, porosity, and non-woven dressings, etc., where each type of dressings has their respective pros and cons. For powder dressings, they are applicable on wound beds with larger area, however when in contact with fluid, they clot easily to increase the difficulty of removal. Foam dressings are capable of managing the moisture of the wound bed to prevent further breaks on the wounded site. However, foam dressings only react on the surface of the wound bed, thus dealing very limited effect to cavity wounds. For porosity dressings, they are able to absorb a vast amount of wound exudates to prevent the wounds from inflaming, and to maintain the appropriate balance of moisture in the wound environment. However, they are also difficult to remove upon absorbing the wound exudates. Non-woven dressings have bio-macromolecular contents which are compatible to organisms to promote healing. However, the structure of the non-woven dressings are often damaged upon absorbing wound exudates, resulting in easy adherence to the wound bed. Hence, causing rupture of the wound at each dressing change.

Furthermore, please refer to FIG. 1. Generally, the dressings 12 of prior arts are applied directly on the wound W of the skin P However, there are some shortcomings needed to be overcome for the typical dressings 12. For instance, the dressings 12 only react on the surface, without going deep into the wound W. Furthermore, the dressings 12 can not be easily removed from the wound W as they adhere to the skin P easily. Also, the dressings 12 are unable to maintain the appropriate balance of moisture in the wound W environment.

### SUMMARY OF THE INVENTION

The object of the instant disclosure is to provide a dressing for wounds, which can overcome the problems encountered by conventional dressings. For instance, problems such as the incapability of reaching to the interior of the wounds; or maintaining an appropriate balance of moisture in the wound environment, and easily adherence to the skin.

In order to achieve the aforementioned objects, according to an embodiment of the instant disclosure, a wound dressing is provided, which includes a plurality of hydrogel particles and a dressing. Each of the hydrogel particles is a granular colloid which swells upon absorbing the wound exudates, enabling the hydrogel particles to fill up the wound entirely. Furthermore, the dressing covers on the wound to seal the hydrogel particles therein.

A wound dressing which includes a plurality of hydrogel particles and a dressing is further provided. Each of the hydrogel particles is a granular colloid which is capable of releasing the fluid, and the hydrogel particles can fill up the wound entirely. Furthermore, the dressing covers on the wound to seal the hydrogel particles therein.

Based on the above, the hydrogel particles of the instant disclosure will swell upon absorbing the wound exudates. Therefore, the hydrogel particles can fill up the wound entirely. Furthermore, the hydrogel particles are capable of absorbing the exudates to maintain the appropriate balance of moisture in the wound environment. Also, during the recovery of the wounded tissues, the hydrogel particles play the role as a media for the wound to progress toward closure, so as to promote healing. Moreover, the smooth and moist texture of the hydrogel particles can also avoid the dressing from adhering to the wound. And the hydrogel particles can be simply removed by rinsing normal saline directly on the wound, which provides convenience for the healthcare professionals, medical personnel or patients to operate.

In order to further appreciate the characteristics and technical contents of the instant disclosure, references are hereunder made to the detailed descriptions and appended drawings in connection with the instant disclosure. However, the appended drawings are merely shown for exemplary purposes, rather than being used to restrict the scope of the instant disclosure.

### BRIEF DESCRIPTION OF THE DRAWINGS

FIG. 1 shows a schematic view of a wound dressing of the prior art;

FIG. 2 shows a schematic view of a plurality of hydrogel particles of a wound dressing filling up the wound according to the instant disclosure;

FIG. 3 shows a schematic view of a dressing covered on the wound according to the instant disclosure;

FIG. 4 shows a schematic view of a plurality of hydrogel particles of a wound dressing which swells upon absorbing the wound exudates according to the instant disclosure;

FIG. 5 shows a partially enlarged view of FIG. 4;

FIG. 6 shows a schematic view of the removal of the hydrogel particles of the instant disclosure.

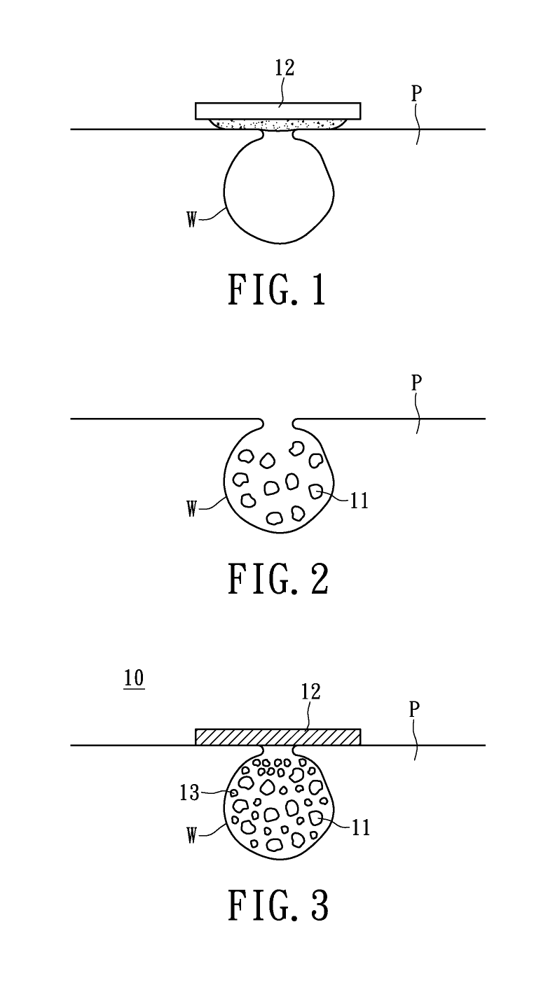

### DETAILED DESCRIPTION OF THE PREFERRED EMBODIMENTS

A wound dressing 10 is provided in the instant disclosure, which includes a plurality of hydrogel particles 11, and a dressing 12. Each of the hydrogel particles 11 is a granular colloid. The dressing 12 covers on the wound W to seal the hydrogel particles 11 within the wound W.

Please refer to FIG. 2. The hydrogel particles 11 of the instant disclosure are disposed into the wound W of the skin P. The method of disposing the hydrogel particles 11 includes, but not limited to, inserting through an injecting tool, pouring in directly, putting in through a clamping tool, inserting through the help of a machine, inserting through a tube-like tool, and inserting through a funnel-like tool, etc. The hydrogel particles 11 are disposed to fill up the wound W entirely, so as to establish a full contact with the wound W.

Please refer to FIG. 3. When the hydrogel particles 11 are completely disposed into the wound W, a wound care agent 13 can be further provided to mix along with the hydrogel particles 11. The dressing 12 is then covered on the wound W to seal the hydrogel particles 11 within the wound W to avoid the hydrogel particle 11 from escaping. Besides, by covering the dressing 12 on the wound W can also absorb the over-produced exudates, and prevent the wound W from infection through bacteria, etc., and other external physical or chemical threats. The dressing 12 can be made of natural fiber, artificial fiber, woven or non-woven, or materials which are suitable to establish contact with the wound W. Furthermore, an additional tool can be utilized to fasten the dressing 12. For instance, a gauze bandage, a woven elastic bandage, a breathable paper tape, a triangular bandage, or other medical appliances can be utilized to fasten the dressing 12. On the other hand, the dressing 12 can also be an adhesive material which can adhere to the skin P. In other words, the dressing 12 can be applied directly to attach on the wound W bed. However, the method of fastening the dressing 12 to the wound W is not restricted thereto.

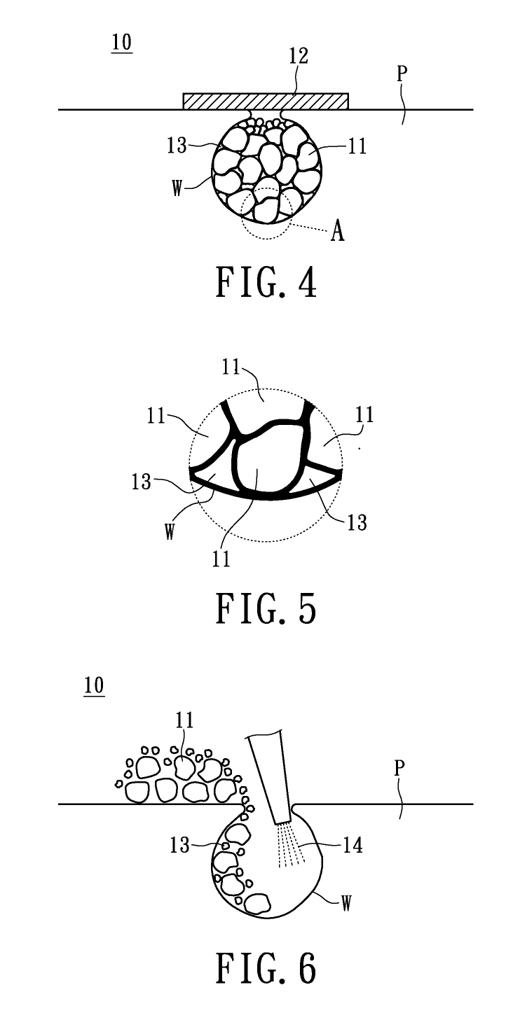

Please refer to FIG. 4. After the hydrogel particles 11 are disposed into the wound W of the skin P, the hydrogel particles 11 will absorb the exudates from the wound W to swell. Thereby, the swelled hydrogel particles 11 will fill up the wound W completely. Furthermore, as the wound W exudates are absorbed by the hydrogel particles 11, therefore, the total volume thereof will increase to expansively establish a tight contact with the wound W. Please refer to FIG. 5, which shows a partially enlarged view of FIG. 4 according to the instant disclosure. Since the hydrogel particles 11 are elastic, they can endure with the squeezing to transform into random shapes, so as to establish a tighter contact with the wound W, and to fill up the wound W in a gapless manner. Furthermore, the ability of transformation also enables the hydrogel particles 11 to adapt to wounds W of different shapes and irregular inner cell walls. Comparing to wound dressings of the prior art, the wound dressing 10 of the instant disclosure can establish a better contact with the wounds W.

Due to the swelling and transforming characteristics of the hydrogel particles 11, they can be adapt to wounds W of different shapes. Furthermore, the hydrogel particles 11 can be utilized to fill up other parts of the body where holes are formed, even if the hole has a relatively small opening. For instance, the hydrogel particles 11 can be utilized in dental medications or surgical operations for decayed teeth or defect tissues. The hydrogel particles 11 can be also inserted within the body to act as a buffering material. Therefore, the hydrogel particles 11 are capable of filling up the tissues having defects within the body. Furthermore, the hydrogel particles 11 can also be applied on non-human body tissues to fill up the holes within other organisms. Similarly, the hydrogel particles 11 can absorb liquid to swell, so as to fill up the hole entirely, or release fluid to keep the hole moist.

Other than that, the hydrogel particles 11 can also act as a media to promote healing. During the natural process of dermal wound W healing, the hydrogel particles 11 serve to guide the necessary cells, including an endogenous mixture of growth factors, to migrate to the wound W site. These macromolecules will merge to combine and provide the basis for granulation tissue formation, enabling the wound W to progress toward closure. Thereby, the hydrogel particles 11 can effectively promote healing.

The hydrogel particles 11 may also be granular colloids capable of releasing the fluid. When fluid within the wound W decreases or the wound W is unable to produce enough exudates, the wound W will gradually dry out. The hydrogel particles 11 will then release the fluid therein to moist the wound W. More specifically, as a certain amount of liquid is contained in the hydrogel particles 11, therefore, the hydrogel particles 11 is capable of releasing a sufficient amount of fluid to moist the wound W even without absorbing wound W exudates. Based on the above, the hydrogel particles 11 are capable of maintaining the appropriate balance of moisture in the wound W environment. For instance, when excess exudates are produced by the wound W, the hydrogel particles 11 will absorb the exudates to prevent bacterial proliferation or avoid maceration of the surrounding healthy tissue; conversely, when the wound W gradually dries out, the hydrogel particles 11 will release the fluid therein to keep the wound W under a moist environment to promote healing. Therefore, the hydrogel particles 11 are capable of managing fluid in wounds W to maintain an optimal healing environment.

In addition, to achieve a better healing effect for the wound dressing 10 of the instant disclosure, the wound care agent 13 can be further provided by mixing along with the hydrogel particles 11, where the wound care agent 13 includes anti-inflammatory agent, pain relieving agent, growth hormone, antibiotics, anti-infective agent, and/or other drugs which promote healing. Therefore, the hydrogel particles 11 can achieve a better healing effect with a decreased infection possibility for the wound W through the addition of the wound care agent 13.

Please refer to FIG. 6. If the hydrogel particles 11 are required to be removed from the wound W, a removal agent 14 can be used to perform the removing process. The removal agent 14 can be normal saline, distilled water, pure water, or other liquid which is friendly and suitable for humans. Due to the smooth and moist texture of the hydrogel particles 11, they are uneasy to adhere to the cell walls within the wound W. Therefore, the removal agent 14 can be injected directly into the wound W to wash away the hydrogel particles 11. Thereby, providing convenience for the healthcare professionals, medical personnel, and the patients to operate.

Based on the above, the instant disclosure has the following advantages:

1. The hydrogel particles of the instant disclosure will swell upon absorbing wound exudates to fill up the wound. Furthermore, the hydrogel particles having capability of absorbing and releasing the fluid therein can maintain an appropriate balance of moisture in the wound environment.

2. During the proliferation of the cell types in dermal wound healing, the hydrogel particles will play the role as a media for the cells to merge, and eventually toward the closure of the wound. Thereby, promoting the healing effect of the wound.

3. Due to the smooth and moist texture of the hydrogel particles, they are uneasy to adhere to the cell walls within the wound. Therefore, normal saline can be injected directly into the wound to wash away the hydrogel particles. Thereby, providing convenience for the healthcare professionals, medical personnel, and the patients to operate.

The descriptions illustrated supra set forth simply the preferred embodiments of the instant disclosure; however, the characteristics of the instant disclosure are by no means restricted thereto. All changes, alternations, or modifications conveniently considered by those skilled in the art are deemed to be encompassed within the scope of the instant disclosure delineated by the following claims.

What is claimed is:

1. A wound dressing, comprising:
a plurality of hydrogel particles, wherein each of the hydrogel particles is a granular colloid which swells upon absorbing fluid, wherein the hydrogel particles fill up a wound entirely; and
a dressing covered on the wound to seal the plurality of hydrogel particles within the wound.

2. The wound dressing according to claim 1, wherein the dressing is made from a material selected from the group consisting of natural fiber, synthetic fiber, woven material, and non-woven material.

3. The wound dressing according to claim 1, further comprising a wound care agent mixed along with the hydrogel particles.

4. The wound dressing according to claim 3, wherein the wound care agent includes anti-inflammatory agent, pain relieving agent, growth hormone, antibiotics, anti-infective agent, and other wound healing drugs.

5. The wound dressing according to claim 1, wherein the hydrogel particles are removed from the wound through a removal agent, wherein the removal agent is selected from the group consisting of normal saline, distilled water, and pure water.

6. A wound dressing, comprising:
a plurality of hydrogel particles, wherein each of the hydrogel particles is a granular colloid capable of releasing the fluid, wherein the hydrogel particles fill up a wound entirely; and
a dressing covered on the wound to seal the plurality of hydrogel particles within the wound.

7. The wound dressing according to claim 6, wherein the dressing is made of a material selected from the group consisting of natural fiber, synthetic fiber, woven material, and non-woven material.

8. The wound dressing according to claim 6, further comprising a wound care agent mixed along with the hydrogel particles.

9. The wound dressing according to claim 8, wherein the wound care agent includes anti-inflammatory agent, pain relieving agent, growth hormone, antibiotics, anti-infective agent, and other wound healing drugs.

10. The wound dressing according to claim 6, wherein the hydrogel particles are removed from the wound through a removal agent, wherein the removal agent is selected from the group consisting of normal saline, distilled water, or pure water.
